(19) United States
(12) Patent Application Publication
(10) Pub. No.: US 2022/0380552 A1
(43) Pub. Date: Dec. 1, 2022

(54) SILICONE OIL TERPOLYMER FOR USE IN INTRAOCULAR LENS DEVICES

(71) Applicant: LensGen, Inc., Irvine, CA (US)

(72) Inventors: Thomas A. Silvestrini, Alamo, CA (US); Kevin Yacoub, Irvine, CA (US)

(21) Appl. No.: 17/755,773

(22) PCT Filed: Nov. 5, 2020
(86) PCT No.: PCT/US2020/059177

§ 371 Date: May 6, 2022

Related U.S. Application Data
(60) Provisional application No. 62/932,033, filed on Nov. 7, 2019.

(51) Int. Cl. C08G 77/24; A61F 2/16

(52) U.S. Cl. CPC C08G 77/24; A61F 2/1635; A61F 2/1648; A61F 2002/1682; A61F 2002/16901

(57) ABSTRACT

A halosilicone oil in the form of an uncrosslinked terpolymer represented by the following Formula (I):

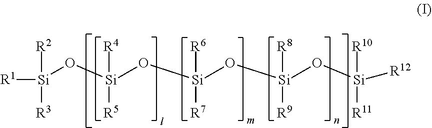

is disclosed, wherein at least one block forming component includes a halogenated, preferably fluorinated, substituent such as 2,2,2-trifluoroethyl or 3,3,3-trifluoropropyl. In some implementations, at least one block forming component also includes an aryl group, such as phenyl, that raises the refractive index of the polymer. In some implementations, the halosilicone oil is incorporated into an intraocular lens.

### CROSS-REFERENCE TO RELATED APPLICATIONS

This application claims the benefit of U.S. Provisional Application No. 62/932,033, filed on Nov.7, 2019, which is hereby incorporated by reference in its entirety.

### FIELD OF THE INVENTION

The invention relates generally to silicone oil, and more particularly to silicone oil suitable for use in intraocular lens devices.

### BACKGROUND

Surgical procedures on the eye have been on the rise as technological advances permit for sophisticated interventions to address a wide variety of ophthalmic conditions. Patient acceptance has increased over the last twenty years, as such procedures have proven to be generally safe and produce results that significantly improve patient quality of life.

Cataract surgery remains one of the most common surgical procedures, with over 16 million cataract procedures being performed worldwide. It is expected that this number will continue to rise as average life expectancies continue to increase. Cataracts are typically treated by removing the crystalline lens from the eye and implanting an intraocular lens (“IOL”) in its place. Conventional IOL devices typically provide vision correction at only a single distance via a monofocal lens, and thus fail to correct for presbyopia. Accordingly, while patients who undergo a standard IOL implantation no longer experience clouding from cataracts, they are unable to accommodate, or change focus from near to far, from far to near, and to distances in between, and still require use of corrective glasses.

Surgeries to correct refractive errors of the eye have also become extremely common, of which LASIK enjoys substantial popularity with over 700,000 procedures being performed per year. Given the high prevalence of refractive errors and the relative safety and effectiveness of this procedure, more and more people are expected to tum to LASIK or other surgical procedures over conventional eyeglasses or contact lenses. Despite the success of LASIK in treating myopia, there remains an unmet need for an effective surgical intervention to correct for presbyopia, which cannot be treated by conventional LASIK procedures.

As nearly every cataract patient also suffers from presbyopia, there is convergence of market demands for the treatment of both these conditions. Various modifications of IOL devices have been introduced to address ophthalmic cataracts and/or presbyopia in patients. For instance, multifocal lenses for IOL devices were introduced to provide vision correction at more than one distance with the goal of obviating the need for additional corrective lenses required with the monofocal lenses. Multifocal lenses generally have areas of varying refractive power to provide vision at multiple distances (e.g., near, intermediate and far). However, one significant disadvantage to multifocal lenses is the possibility of visual distortions, particularly in the form of glare and halos around light sources at night.

Accommodating IOL devices have also been recently introduced for use in cataract surgery. Accommodating IOL devices often feature a monofocal lens configured to move and/or change shape in response to the eye's natural mechanism of accommodation, thereby providing vision correction over a broad range of distances. Such accommodating IOL devices may also feature a haptic system protruding from the central lens. Such haptic systems are typically configured to respond to the contraction and relaxation of the eye's ciliary muscles and ultimately effect changes in the central lens to provide varying diopters of power.

Some IOL devices may also include a fluid therein, where the movement of said fluid may result in an optical power change. However, conventional fluids have been found to lead to undesirable swelling of the bulk polymer material(s) comprising the IOL device (e.g., the lens, the haptic system, etc.). There is therefore a need to develop improved fluids for use in IOL devices that minimize or eliminate the swelling of the bulk polymer material(s) of said devices.

### SUMMARY

in certain implementations, an intraocular lens (IOL) device is provided. The IOL device may comprise a power changing lens comprising a first side; a second side; and a peripheral portion extending between and connecting the first and second sides; wherein the first side, second side and peripheral portion form a closed cavity at least partially filled by an uncrosslinked fluorosilicone terpolymer fluid.

In certain implementations, the fluid is a halogenated silicone oil according to Formula (I)

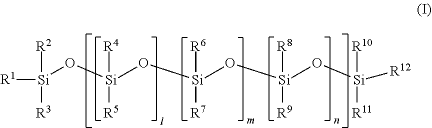

wherein R¹ and R¹² are independently selected from optionally substituted alkyl or optionally substituted alkenyl including vinyl(alkyl); each of R², R³, R¹⁰ and R¹¹ may be independently hydrogen or optionally substituted alkyl; R⁴ is optionally substituted haloalkyl, including fluoroalkyl; R⁵ is optionally substituted alkyl or optionally substituted haloalkyl, including fluoroalkyl; R⁶ is optionally substituted aryl or optionally substituted aryl(alkyl); R⁷ is optionally substituted aryl, optionally substituted aryl(alkyl), or optionally substituted alkyl; each of R⁸ and R⁹ are independently optionally substituted alkyl; 1 is a molar fraction of 0.01 to 0.8; m is a molar fraction of 0.01 to 0.5; and n is a molar fraction of 0.01 to 0.6. The formula above refers to a random block copolymer in with the blocks are made up of multiple —Si(R^{X}, R^{X})O— silicone units of a particular type combined randomly with other blocks of different types. The l, m, and n refer to the total mole fraction of those specific silicone units in the polymer exclusive of the end blocker. The structure above is a formula rather than a literal structure of the polymer; the polymer does not consist of only three large blocks in the order shown.

In some implementations there is a power changing intraocular lens comprising a first side, a second side, and a peripheral portion extending between and connecting the first and second sides wherein the first side, second side and peripheral portion form a closed cavity at least partially filled by an halosilicone fluid or lens oil. In some implementations, the halosilicone fluid has a surface tension at least 0.5 mN/m greater than a surface tension of the first side and a surface tension of the second side. In some implementations, the halosilicone fluid is of Formula (I).

In some implementations there is an accommodating IOL, comprising a base lens, a haptic, and a power changing lens. The power changing lens may comprise a first side, a second side, a peripheral portion coupling the first and second sides, and a closed cavity configured to house a fluid such as a silicone lens oil as disclosed herein. The power changing lens may be spaced from a first edge of the haptic. In some implementations, the haptic comprises a first open end, a second end coupled with the base lens, and an outer periphery configured to engage a capsular bag, including the equatorial region of the capsular bag. The haptic may further include an inner periphery and a height between a first edge and a second edge, the inner periphery disposed about a cavity and having a lens retention portion configured to receive and retain a lens.

Other objects, features and advantages of the described compounds and devices will become apparent to those skilled in the art from the following detailed description. It is to be understood, however, that the detailed description and specific examples, while indicating preferred implementations, are given by way of illustration and not limitation. Many changes and modifications may be made without departing from the spirit thereof, and the invention includes all such modifications.

### BRIEF DESCRIPTION OF THE DRAWINGS

Preferred and non-limiting implementations may be more readily understood by referring to the accompanying drawings in which:

FIGS. 1A and 1B are sectional views illustrating certain anatomical features of the human eye with an intraocular lens device implanted in the lens capsule thereof, where the IOL device is in an accommodated and unaccommodated state, respectively.

FIG. 2 is an anterior perspective view of the accommodating IOL device shown in FIG. 1;

FIG. 2A is an anterior view of the accommodating IOL device of FIG. 2;

FIG. 2B is an exploded view of the accommodating IOL device of FIG. 2;

FIG. 2C is a cross-sectional view of the accommodating IOL device of FIG. 2 taken at section plane 2C-2C;

FIG. 2D is a cross-sectional view of the accommodating IOL device of FIG. 2 taken at section plane 2D-2D;

FIG. 3 is a cross-sectional view of a power changing lens of the accommodating IOL of FIG. 2 taken at section plane 2C-2C;

FIG. 4 are optical coherence tomography (OCT) images showing how the deflection of a surface of a silicone IOL changes depending upon the makeup of the fluid contained within the IOL.

FIG. 5 shows details of GPC results of two examples of terpolymer according to the examples E1 herein.

### DETAILED DESCRIPTION OF THE PREFERRED EMBODIMENTS

Specific, non-limiting implementations of the lens oil and IOLs including the lens oil will now be described. It should be understood that particular features and aspects of any embodiment or implementation disclosed herein may be used and/or combined with particular features and aspects of any other embodiment or implementation disclosed herein. It should also be understood that such embodiments are by way of example and are merely illustrative of but a small number of implementations within the scope of the present disclosure.

### Definitions

The following definitions are used in relation to the discussion of the terpolymer of Formula (I) and related terpolymers. Whenever a group is described as being “substituted” or “optionally substituted” that group may be substituted with one or more of the indicated substituents. If no substituents are indicated, it is meant that the indicated “optionally substituted” or “substituted” group may be substituted with one or more group(s) individually and independently selected from alkyl, alkenyl, hydroxy, hydroxyalkyl, alkoxy, aryl, heteroaryl, aryl(alkyl), heteroaryl(alkyl), halogen, and haloalkyl.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms or 1 to 4 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

“Alkylene groups” are straight-chained —CH₂— tethering groups, forming bonds to connect molecular fragments via their terminal carbon atoms. Examples include but are not limited to methylene (—CH₂—), ethylene (—CH₂CH₂—) propylene (—CH₂CH₂CH₂—), and butylene (—CH₂CH₂CH₂CH₂—). An alkylene group can be substituted by replacing one or more hydrogen of the alkylene group with a substituent(s) listed under the definition of “substituted.” An alkylene group can have 1 to 20 carbons, including I to 6 carbons or I to 4 carbons. Groups having 6 or fewer carbons may also he referred to as “lower” alkylene groups.

As used herein, “alkenyl” refers to a straight or branched hydrocarbon chain having one or more double bonds. Examples of alkenyl groups include vinyl, vinylmethyl and ethenyl. An alkenyl group may he unsubstituted or substituted. As used herein, “vinyl(alkyl) refers to an alkenyl group in which a terminal vinyl group is connected, as a substituent, via an alkylene group, including a lower alkylene group.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, phenyl and naphthyl. An aryl group may be substituted or unsubstituted.

As used herein, “heteroaryl” refers to a monocyclic or multicyclic aromatic ring system (a ring system with fully delocalized pi-electron system) that contain(s) one, two, three or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinohne, isoquinoline, quinazoline, quinoxaline, cinnoline and triazine. A heteroaryl group may be substituted or unsubstituted.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, xylyl, tolyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group The lower alkylene and heteroaryl group of heteroaralkyl may he substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl and 2-fluoroisobutyl. A haloalkyl may be substituted or unsubstituted. The term “halogen atom” or “halogen” or “halo” as used herein, means any one of the radio-stable atoms of column 7 of the Periodic Table of the Elements, such as, fluorine, chlorine, bromine and iodine.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiornerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

### Implanted Intraocular Lens Device

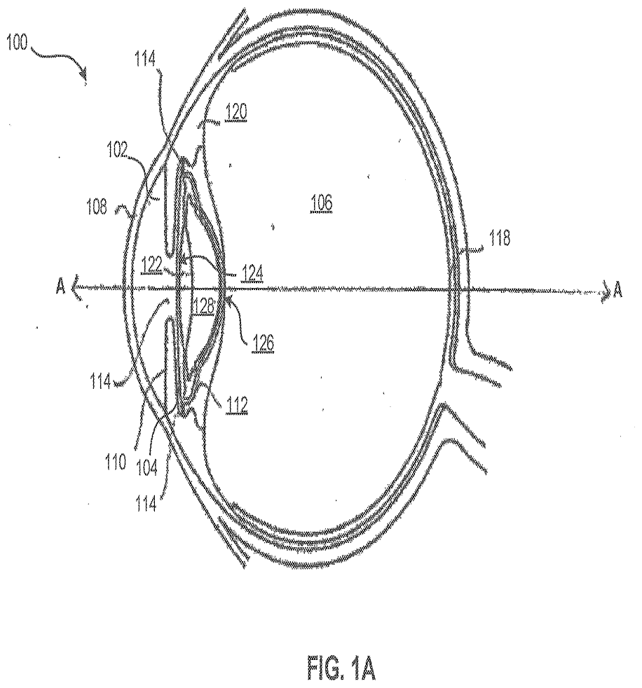

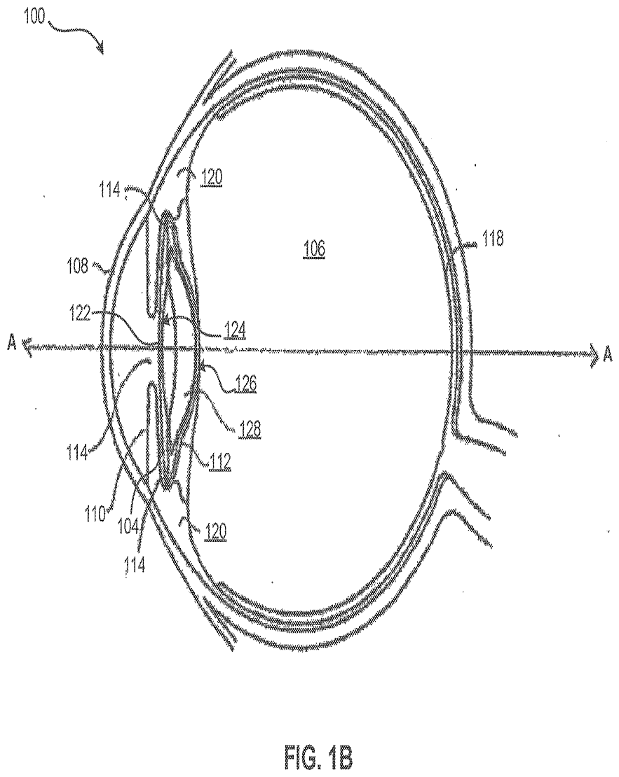

FIGS. 1A-1B illustrate a simplified schematic of a human eye, and a intraocular lens (IOL) device implanted in the lens capsule thereof. As shown in FIGS. 1A-1B, the human eye 100 comprises three fluid-filled chambers: the anterior chamber 102, the posterior chamber 104, and the vitreous chamber 106. The anterior chamber 102 generally corresponds to the region between the cornea 108 and the iris 110, whereas the posterior chamber 104 generally corresponds to the region bounded by the iris 110, the lens capsule 112, and the zonule fibers 114 connected to the lens capsule 112. The anterior and posterior chambers 102, 104 contain the aqueous humor, a fluid which flows therebetween through the pupil 116 (an opening defined by the iris 110). Light enters the eye 100 through the pupil 116 and travels along the visual axis A-A, ultimately striking the retina 118 to produce vision. The amount of light entering the eye 100 is directly related to the size of the pupil 116, which is regulated by the iris 110.

The vitreous chamber 106 generally corresponds to the region between the lens capsule 112 and the retina 118. The vitreous chamber 106 contains the vitreous fluid, a transparent, colorless, gelatinous mass that is more viscous than the aqueous humor. Although much of the volume of the vitreous humor is water, it also contains cells, salts, sugars, vitrosin, a network of collagen type II fibers with glycosaminoglycan hyaluronic acid, and proteins. Preferably, the vitreous has a viscosity that is two to four times that of pure water, giving it a gelatinous consistency. The vitreous humor may also have a refractive index of 1.336.

The lens capsule 112 typically houses the eye's natural lens (not shown). The natural lens is an elastic, clear, crystalline membrane-like structure maintained under tension via the ciliary muscles 120 and zonule fibers 114. As a result, the natural lens tends to have a rounder configuration, a shape it must assume for the eye 100 to focus at a near distance. Changing the shape of the natural lens alters the focus distance of the eye. Accordingly, the eye's natural mechanism of accommodation is reflected by changes in the shape of said lens.

To correct for ophthalmic cataracts and/or refractive errors such as presbyopia, the natural lens housed in the lens capsule 112 may be removed and replaced with an IOL, device 122. Implantation of the IOL device 122 may be accomplished by first removing the natural lens housed within the lens capsule 112 through a small incision using standard surgical procedures, such as phacoemulsification. After removal of the natural lens, the IOL device 122 may then be introduced into the lens capsule 112 through the small incision.

As shown in the non-limiting embodiment of FIGS. 1A-1B, the IOL device 122 may be characterized as having an anterior region 124 facing the posterior chamber 104 of the eye 100. The anterior region 124 of the IOL device 122 may include a refractive optical element (not shown) centered about the optical axis A-A. The IOL device 122 may also be characterized as having a posterior region 126 coupled to the anterior region 124, with the posterior region 126 facing the vitreous chamber 106 of the eye 100. The IOL device 122 may additionally have a cavity region 128 defined between the anterior and posterior regions 124, 126, in which a fluid (e.g., a lens oil) may be disposed. In some aspects, the fluid may be introduced into the cavity region 128 through a self-sealing valve in the IOL device 122 after implantation of the IOL device 122 in the lens capsule 112. The volume of the fluid contained within the IOL device 122 may be tailored according to the size of the lens capsule 112 for each patient, as would be appreciated by skilled artisans upon reading the present disclosure. In preferred aspects, the volume of the fluid in the cavity region 128 may be sufficient so as to permit engagement of a peripheral region 130 of the TOT, device 122 with the zonule fibers 114 and ciliary muscles 120.

Similar to the natural lens, the IOL device 122 changes its shape in response to the accommodative mechanisms of the eye 100. For instance, FIG. 1A shows the eye 100 in a generally accommodated state, as may be the case when the eye 100 is focusing on a nearby object. In such an accommodated state, the ciliary muscles 120 contract and move in an anterior direction. The contraction of the ciliary muscles 120 reduces the stress exerted on the zonule fibers 114, which in turn reduces the stress exerted by the zonule fibers 114 on the lens capsule 112. As a result, the IOL device 122 undergoes elastic recovery and may achieve a more rounded, biconvex shape.

FIG. 1B shows the eye 100 in a generally unaccommodated state, as may be the case when the eye 100 is focusing at a distance. In such an unaccommodated state, the ciliary muscles 120 relax, thereby increasing the diameter of its opening and causing the zonule fibers 114 to pull away from the optical axis A-A. This, in turn, causes the zonule fibers 114 to radially pull on the periphery of the lens capsule 112, which causes the IOL device 122 to assume a flatter shape/geometry as compared to the accommodated state. The flatter shape/geometry of the lens capsule 112, and the IOL device 122 disposed therein, corresponds to a reduction in the ability to bend or refract light entering the pupil 116.

### Intraocular Lens Device

Intraocular lens (IOL) devices suitable for implantation in the lens capsule of a patient's eye may include those described in U.S. Pat. No. 9,186,244, issued Nov. 17, 2015; U.S. Patent Application Publication No. 2013/0053954, published on Feb. 28, 2013; U.S. Patent Application Publication No. 2016/0030161, published on Feb. 4, 2014; U.S. patent application Ser. No. 15/144,544, filed on May 2, 2016; U.S. patent application Ser. No. 15/144,568, filed on May 2, 2016; International Patent Application Publication No. WO 2016/049059, published on Mar. 31, 2016, and International Patent Publication No. WO 2019/236908, published Dec. 12, 2019, the disclosures of which are incorporated herein for reference in their entireties. It is understood that the silicone oil may be incorporated in the IOL devices described in the foregoing patent references. For example, the silicone oil described herein can be used in place of the fluid described in any of these patent references.

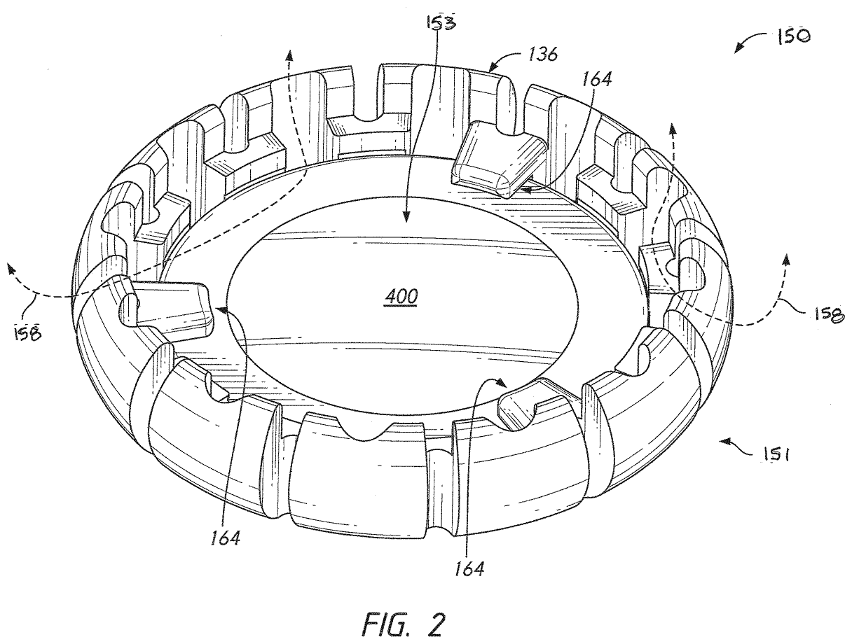

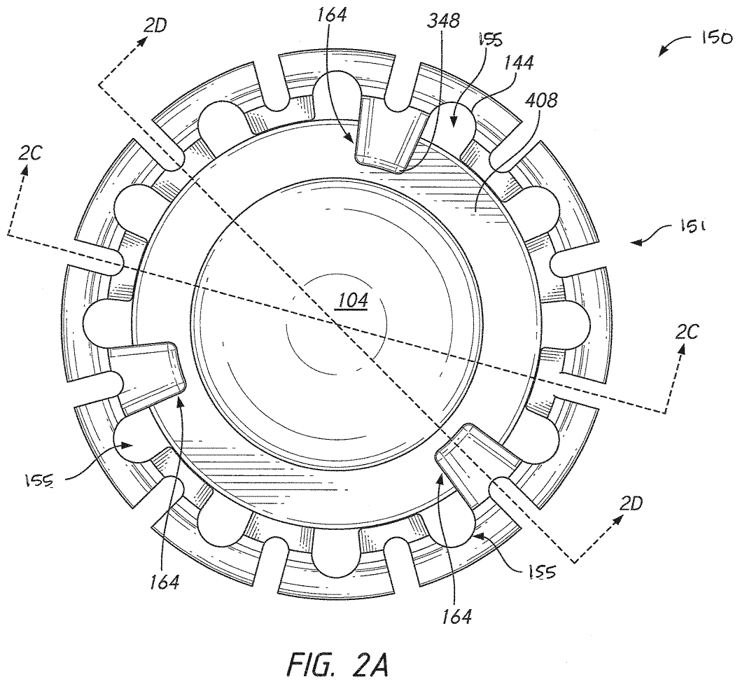

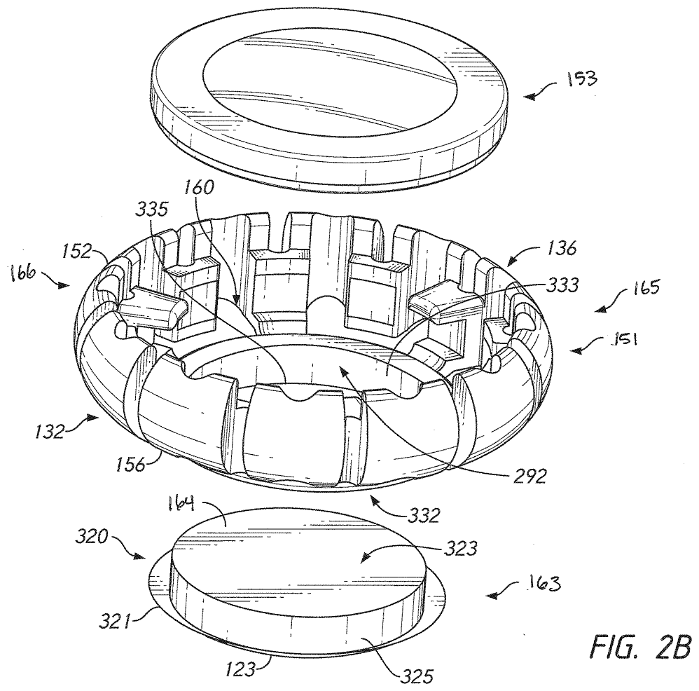

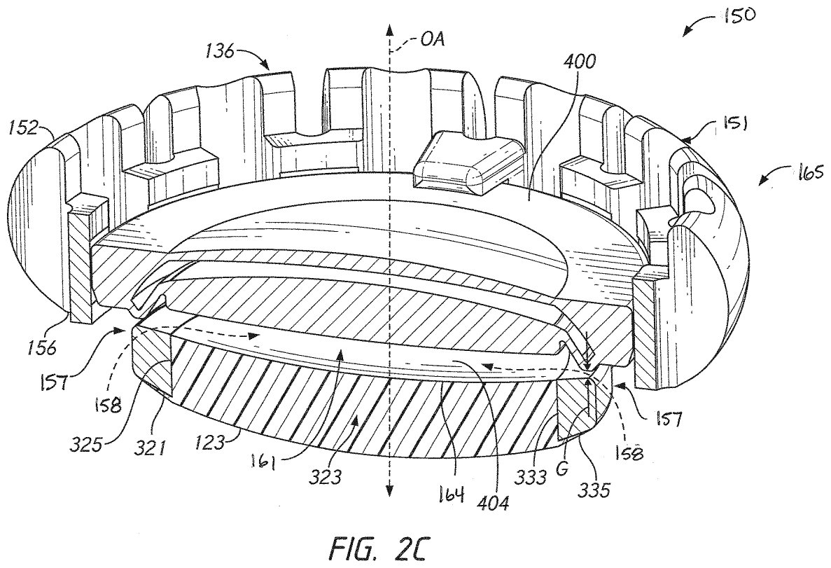

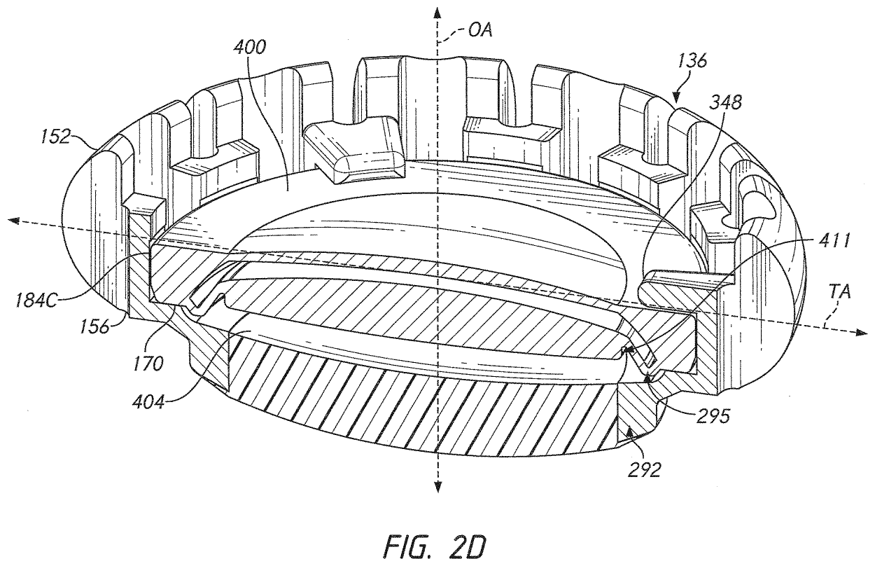

In certain preferred implementations, the silicone oils described herein may be incorporated in an IOL device as described below and illustrated in FIGS. 2-2D.

FIGS. 2-2D show variants of the accommodating IOL device 150. The accommodating IOL device 150 includes a base member 151 and a power changing lens 153. The power changing lens 153 is separate from the base member 151 such that the base member 151 and the power changing lens 153 can be delivered separately, e.g., sequentially. The base member 151 can be delivered before the power changing lens 153. The power changing lens 153 can be subsequently delivered into the base member 151 and can be unfolded within the base member 151 when the base member 151 is in the capsular bag of an eye. This sequential delivery allows the base member 151 and the power changing lens 153 to have more complex structure, providing premium function and yet still be deliverable through a small incision.

The base member 151 can include a base lens 163 and a haptic 165. The base lens 163, if present, provides some of the focusing power of the accommodating IOL device 150. The haptic 165 extends into the equatorial region of the capsular bag and establishes a mounting position for the power changing lens 153. In one embodiment the base lens 163 and the haptic 165 cooperate to maintain the capsular bag in an expanded state, similar to the shape and size of the crystalline lens 54 prior to the capsulotomy. As such, the base member 151 is large compared to traditional non-premium IOLs which are designed for delivery through a small incision.

The power changing lens 153 includes multiple optical components, e.g., a membrane at a first side 400 and a lens at a second side 404. An optical fluid can be disposed between the first side 400 and the second side 404 The power changing lens 153 includes a peripheral portion 408 that together with the optical components at the first side 400 and second side 404 contain the optical fluid. The optical fluid has a number of advantages, including transferring compressive forces from the peripheral portion 408 of the power changing lens 153 to the deformable optical surfaces in a controlled manner to provide optically acceptable surfaces across the range of accommodation. The structure of the power changing lens 153 is much more complex than a traditional non-premium IOL, to provide premium function.

Separating the base member 151 and the power changing lens 153 prior to insertion into the eye enables a smaller incision size than were all the optical components inserted simultaneously as unit. The base member 151 and the power changing lens 153 can be compressed to a greater extent when separate than when they are combined into an assembly. Additionally, forming the base member 151 separate from the power changing lens 153 enables the base member 151 to be used in other IOL devices that may not necessarily be accommodating.

FIG. 2A shows that the accommodating IOL device 150 can have one or a plurality of open channels 155 when assembled. The open channels 155 extend in an anterior and posterior direction between a posterior side of the base member 151 and an anterior side of the power changing lens 153. There are twelve open channels 155 in the illustrated embodiment. There can be more or fewer open channels 155, e.g., at least two, at least four, at least six, at least eight, or at least ten open channels 155. Preferably there are an even number of channels arranged symmetrically relative to an optical axis A of the IOL device 150. Each of the open channels 155 is defined in part by an inner periphery 144 of the base member 151 and by the outer periphery or outer peripheral portion 408 of the power changing lens 153. The open channels 155 can each be disposed between adjacent compression arms 180 of the base member 151, which are discussed further below. The open channels 155 allow fluid to circulate in the capsular bag, e.g., to flow between anterior and posterior sides of the device 150 and to flow from areas outside the optical zone of the accommodating IOL device 150 to within the optical zone. This fluid flow can reduce the tendency of the accommodating IOL device 150 to create pressurized zones between the capsular bag and the surfaces of the components of the device 150.

FIG. 2C shows the accommodating IOL device 150 can have one or a plurality of open channels 157 when assembled that can also provide for flow from outside the accommodating IOL device 150 into a space 161 disposed between the base member 151 and the power changing lens 153. FIG. 2C shows fluid flow 158 that can be provided through the open channels 157. As discussed further below, the channels 157 are configured to permit fluid flow but to restrict migration of cells into the space 161. The channels 155 are provided in a posterior-anterior direction to provide or enhance flow 158 between the anterior and posterior sides of the accommodating IOL device 150 as shown in FIGS. 2 and 2A. The plurality of open channels 157 can allow fluid to flow from outside the accommodating IOL device 150 to a location between the base lens 163, if present, and the second side 404 of the power changing lens 153. By opening the space 161 to flow of fluid from outside the accommodating IOL device 150 to a location between the base lens 163 and the second side 404, the application of force in a direction transverse to the optical axis OA rather than along the optical axis OA is the primary cause of power change. The transfer of forces from the equatorial region 74 through the base member 151 to the power changing lens 153 can be uniquely configured to cause accommodation upon uniformly dispersed radial and circumferential compression.

FIG. 2B shows that the base member 151 has a cavity 160 configured to receive and retain the power changing lens 153. The cavity 160 is defined between the base lens 163 and an opening 136 at an opposite end of the base member 151 and is surrounded by a haptic 165 disposed at a periphery of the accommodating IOL device 150. More specifically, the base lens 163 includes an anterior surface 164 that faces the cavity 160. The anterior surface 164 partly bounds the cavity 160. The base lens 163 also has a posterior surface 123 that faces toward and may contact an anterior side of the posterior portion of the capsular bag. In some embodiments, a curvature of the anterior surface 164 can be lesser than a curvature of posterior surface 123. For example, the curvature of the anterior surface 164 can be less than or equal to about 15 mm⁻¹. The curvature of the anterior surface 164 can be greater than 0 and less than or equal to about 12 mm⁻¹, greater than 0 and less than or equal to about 10 m⁻¹, greater than 0 and less than or equal to about 7 mm⁻¹, greater than 0 and less than or equal to about 5 min⁻¹, greater than 0 and less than or equal to about 3 min⁻¹, or any value in a range/sub-range defined by any of these values. As another example, the base lens 163 can be configured as a plano-convex lens having a substantially planar anterior surface 164. In such embodiments, the curvature of the anterior surface 164 can be 0 or substantially equal to 0 (e.g., less than or equal to 0.1 mm⁻¹. Also, the haptic 165 has a first end 166 and a second end 132 opposite the first end 166. The first end 166 is the end of the haptic 165 that is anterior when the base member 151 is placed in the capsular bag. The second end 132 is the end of the haptic 165 that is posterior to the first end 166 when the base member 151 is placed in the capsular bag. The cavity 160 is disposed between a first edge 152 and a second edge of the haptic 165. As discussed further below, the haptic 165 has anterior and posterior zones disposed about the cavity 160 that are separately configured for retention and compression of the power changing lens 153 and for enhancing overall compressibility of the base member 151.

FIG. 2 shows the power changing lens 153 is inset in the cavity 160. As discussed herein, this state can be achieved in the eye in order to reduce or minimize incision size. A first side 400 of the power changing lens 153 is posterior to an opening 136 of the haptic 165 formed at the first end 166 thereof The configuration of the haptic 165 of the base member 151 assures that the posterior side of the anterior portion of the capsular bag remains spaced away from the posterior portion of the capsular bag. The spacing of the two layers of the capsular bag from each other reduces, minimizes, or eliminates fibrosis between these structures which would limit or reduce the potential for accommodative amplitude. A height 148 of the haptic 165 between a first edge and a second edge of it outer periphery is configured to retain the capsular bag in an open configuration. For example, the height 148 of the haptic 165 can be greater than or equal to about 2 mm. In various embodiments, the height 148 of the haptic 165 can be greater than or equal to about 2.0 mm and less than or equal to about 3.5 mm, greater than or equal to about 2.2 mm and less than or equal to about 3.3 mm, greater than or equal to about 2.5 mm and less than or equal to about 3.0 mm, or any height in a range/sub-range defined by any of these values. The height 148 and profile of the outer periphery 140 are configured to keep anterior portions of the capsular bag anterior of posterior portions of the capsular bag. This can reduce, eliminate or minimize fibrosis or “shrink-wrapping” of the capsular bag. The height 148 and profile of the outer periphery 140 are configured to keep anterior portions of the capsular bag anterior of the power changing lens 153. This can prevent the capsular bag from interfering with the accommodating performance of the accommodating IOL device 150, as discussed further below. In various embodiments, the haptic 165 can comprise an opaque dye (e.g., dark blue dye, indigo dye, violet dye) to increase the visibility of the haptic during implantation of the power changing lens.

The distance from the opening 136 to the first side 400 of the power changing lens 153 and the configuration of the first end 166 of the haptic 165 provide that the anterior portion of the capsular bag remains spaced away from the power changing lens 153. If the anterior portion of the capsular bag were in contact with the first side 400, the accommodating effect of the power changing lens 153 would be reduced, In various embodiments, the distance from the opening 136 to the first side 400 of the power changing lens 153 can be greater than or equal to about 0.6 mm and less than or equal to about 0.75 mm. In various embodiments, the distance from the opening 136 to the first side 400 of the power changing lens 153 can be about 0.01% of the axial height 148 of the haptic 165 to about 37% of the axial height 148 of the haptic 165, Positioning the power changing lens 153 at a distance of about 0.01% of the axial height 148 of the haptic 165 to about 37% of the axial height 148 of the haptic 165, from the opening 136 can advantageously reduce the risk of retinal detachment and PCO as a result of filling the capsular bag. The base member 151 alone and in combination with various second lenses disclosed herein can have a stable effective lens placement (ELP) and/or reduced post-implantation tilt or rotation issues as a result of filling or maintaining the volume of the natural capsular bag. Filling or maintaining the volume of the natural capsular bag can also result in stable refraction after implantation and/or reduced vitreo-retinal tension. Without ascribing to a particular theory, it is believed that by substantially maintaining the volume of the natural capsular bag the vitreous is prevented from shifting anteriorly. Positioning the power changing lens 153 at a distance of about 0.01% of the axial height 148 of the haptic 165 to about 37% of the axial height 148 of the haptic 165, from the opening 136 can advantageously reduce inflammation after surgery.

The accommodating IOL device 150 includes a lens retention portion 164 configured to maintain the power changing lens 153 in an inset position within the cavity 160. The lens retention portion 164 is spaced away from the opening 136 into the cavity 160 of the haptic 165. The lens retention portion 164 can include a plurality of members, as discussed in greater detail below in connection with several figures.

The general structure of the base member 151 is discussed above. FIG. 2B illustrates various additional advantageous aspects of the base member 151.

FIG. 2B shows that the base lens 163 and the haptic 165 can be formed separately and then assembled to form base member 151. In other embodiments the base member 151 is a single molded component with a monolithic structure. The haptic 165 can include the outer periphery 140 configured to contact the equatorial region 74 of the capsular bag and the inner periphery 144 disposed inward of the outer periphery 140 as discussed above. In various implementations, the haptic 165 also can include a lens interface portion, in one example a ring 292, disposed radially inwardly of the inner periphery 144. The ring 292 can be disposed posteriorly of equator contact segments 141 of the outer periphery 140. The ring 292 can be disposed posteriorly of the second end 132 of the haptic 165. The ring 292 can be disposed posteriorly of the second edge 156 of the haptic 165. The position of the ring 292 relative to the equator contact segments 141 of the outer periphery 140 can be selected to place a posterior aspect of the base member 151 in direct contact with the anterior side of the posterior portion of the capsular bag when the base member 151 is placed in the capsular bag. The distance from the ring 292 or from the base lens 163 along the optical axis coupled therewith can be known and controlled and can be a factor in selection of the power changing lens 153 or of another non-accommodating lens, as discussed below.

FIG. 2B shows that the base lens 163 can be coupled with the haptic 165 at the ring 292 (or other lens interface portion). The base lens 163 can have a haptic interface surface 320, which is one example of a haptic interface portion or a peripheral haptic interface portion, and the lens interface surface or portion 332 can have a ring 292. In the illustrated embodiment the lens interface surface or portion 332 includes an annular area disposed about the inner periphery of the ring 292. The lens interface surface or portion 332 can include a posterior surface of the ring 292. In the illustrated embodiment the haptic interface surface 320 of the base lens 163 can include an annular skirt 321 disposed about the periphery of the base lens 163. The haptic interface surface 320 can be a complete annulus in one embodiment. In other embodiments the haptic interface surface 320 can include a plurality of spaced apart members that are disposed about the circumference of the base lens 163. The base lens 163 can be coupled with the haptic 165 at the surfaces or portions 320, 332 by any suitable means including using adhesives, welding or by interlocking connectors such as interference fit posts and recesses or features that can be snapped together, eliminating adhesives and stress concentrations or materials transformations associated with welding.

Stated differently, the base member 151 of an intraocular lens 150 can be assembled using the method described below, which includes coupling and securing the base member haptic 165 and the base lens 163. The base member haptic 165 can include the lens interface portion 332 at the second end 132 that is opposite the first open end 166. The lens interface portion 332 can include ring 292. The base lens 163 can have a central optical portion 323 and a haptic interface portion 320. The base lens 163 can have a periphery 325, which can be circular or can be a cylindrical surface of the central optical portion 323 that faces away from the optical axis thereof, and that is sized to be inserted into the ring 292 of lens interface portion 332. The haptic interface portion 320 can have an annular skirt 321.

The haptic interface portion 320 of the base lens 151 can be coupled with the lens interface portion 332 of the base member haptic 165. The cylindrical or circular periphery 325 of the base lens 163 can be inserted into the ring 292 of the lens interface portion 332 of the base member haptic 165 such that an anterior surface of the annular skirt 321 is coupled to a posterior surface 335 of the ring 292. In some aspects, the lens interface portion 332 can include an opaque structure (e.g., a blue colored structure) and the base lens 163 can include an optically transmissive structure such that coupling the haptic interface portion 320 with the lens interface portion 332 includes transitioning from optically transmissive to optically opaque at an interface or boundary between the base lens 163 and the base member haptic 165.

In some aspects, the haptic interface portion 320 includes an annular member, e.g., the skirt 321, disposed radially outward of the central optical portion 323 and the lens interface portion 332. includes an annular structure, e.g., the ring 292, disposed at the second end 132 of the base member haptic 165 such that coupling the haptic interface portion 320 to the lens interface portion 332 includes placing an anterior side of the annular member, e.g., the skirt 321, against a posterior side 335 of the annular structure, e.g., the ring 292. In some aspects, the haptic interface portion 320 includes a periphery 325 and the lens interface portion 332 includes an optical axis facing surface 333 facing an optical axis OA of the base lens 163 such that coupling the haptic interface portion 320 to the lens interface portion 332 includes advancing the periphery 325 of the central optical portion 323 along the optical facing surface 333 of the base member haptic 165.

In some aspects, the haptic interface portion 320 includes a first transverse surface, e.g., an anterior-facing surface of the skirt 321, disposed transverse to an optical axis OA of the base lens 163 and a first annular surface, e.g. the periphery 325, disposed about an optical axis OA. The lens interface portion 332 can include a second transverse surface 335 (e.g., the posterior face of the ring 292) and a second annular surface, e.g., the optical facing surface 333 (e.g., the portion facing toward the center of the space in which the base lens 163 is mounted). Coupling the haptic interface portion 320 of the base lens 163 with the lens interface portion 332 of the base member haptic 165 can include disposing the first annular surface 325 at least partially within the second annular surface 333, and disposing the first transverse surface 321 adjacent to the second transvers surface 335.

The base lens 151 can be secured to the base member haptic 165 at the lens interface portion 332 and the haptic interface portion 320. Securing the lens interface portion 332 and the haptic interface portion 320 can include applying an adhesive between an anterior surface of the annular skirt 321 and a posterior surface 335 of the ring 292. Securing the lens interface portion 332 and the haptic interface portion 320 can include applying an adhesive between an inward surface 333 of the ring 292 and an outward surface 325 of the base lens 163. The adhesive used to secure the lens interface portion 332 with the haptic interface portion 320 and the annular skirt 321 with the ring 292 can be the same material used to form the base lens 151, the haptic 165 and/or other components of the intraocular lens 150, which can include the materials described herein. The adhesive can be applied where the circular periphery 325 and the annular skirt 321 meet, which can result in the formation of a trough. The trough can include an area disposed around the location where the. skirt 321 and the periphery 325 meet. The anterior surface of the skirt 321 can be inclined such that the free end thereof is at a higher elevation than the end joined to the periphery 325. This construction helps contain the adhesive during assembly, such that the adhesive is maintained away from the optical surfaces of the base lens 163. The base member haptic 165 can be made of a different material than the base lens 163, but nonetheless, the adhesive used to secure the base member haptic 165 and the base lens 151 can be capable of joining or adhering the two different materials.

By forming the base lens 163 separate from the haptic 165, the base member 151 can benefit from using materials that are adapted for the particular purpose. The base lens 163 can be formed from a material with: high optical quality, high compressibility, low coefficient of friction, beneficial tissue engagement properties for impeding posterior capsule opacification, or with any combination of these material properties. In one embodiment the base lens 163 is formed of silicone, but other materials that could be used include acrylic (e.g., hydrophobic and hydrophilic acrylics). Suitable silicone materials are biocompatible for the haptic 165, including medical grade silicones, where preferably the cured material contains a low, negligible, or medically insignificant volume of compounds extractable by water, saline, or ocular fluids at about 37° C. Certain suitable silicone materials have a Young's modulus when cured of less than 100 psi (about 7×10⁵ Pa), or even less than 50 psi (about 3.5×10⁵ Pa), including 5-50 psi (about 3.5×10-3.5×10⁵ Pa), 10-40 psi (about 7×10-3×10⁵ Pa). and 10-35 psi (about 7×10⁴-205×10⁵ Pa), Examples of suitable silicone materials include, but are not limited to, MED 4805, MED4810, MED4820, MED4830, MED5820, and IMED5830from NuSil®. For the optic examples of suitable silicone materials include, but not limited to, MED 6215, MED6210, MED6219, MED 6233 and MED6820. Another suitable silicone material for forming some or all of the optic or other lens components is disclosed in PCT International Publication No. WO 2016/049059, the content of which is hereby incorporated by reference in its entirety. Suitable optic materials may also include a UV chromophore or UV absorbing group that may be blended with or bonded to a silicone component. In some such materials the UV chromophore or UV absorbing group is substantially non-extractible from the cured lens material by water, saline or ocular fluids at about 37° C. Embodiments of the base lens 163 comprising acrylic can be partially manufactured using molding methods and partially machined. The haptic 165 can be made of a material that is the same as or different from the material of the base lens 163. The haptic 165 can be made of a material that is selected to be selectively stiff or incompressible. As discussed further below, the haptic 165 includes compression arms that preferably transfer a high percentage of force from a radially outward position to a radially inward position to produce a large amount of accommodation in the power changing lens 153 for a unit of ocular force. The material for the haptic 165 can also take into consideration a preference for circumferential compression, low friction coefficient, maintaining bulk properties over a large number of cycles, and other properties. One material suitable for the haptic 165 is silicone, including, but not limited to, the silicone materials listed above for the base lens, but other materials could be used.

In some variations discussed further below the base lens 163 is omitted. The base member 151 can include the ring 292 which can directly contact an annular area of the capsular bag disposed about the optical axis. The ring 292 can be extended further posteriorly to provide the same distance to the equator contact segments 141 or the distance can be varied and taken into account when the overall optical design of the power changing lens 153 is selected.

The haptic 165 is configured to set the position of a lens disposed in the cavity 160. The haptic 165 can be configured to set one more of the anterior-posterior location of one or both of the first side 400 and the second side 404 of the power changing lens 153. The haptic 165 can be configured to set the orientation of one or both of the first side 400 and the second side 404 of the power changing lens 153 relative to the optical axis OA of the accommodating IOL device 150.

The haptic 165 can have a surface or a plurality of surfaces that mate with the power changing lens 153 to set the position of the power changing lens 153 along the optical axis OA of the accommodating IOL device 150. FIG. 2D shows that the second side 404 of the power changing lens 153 is placed into the cavity 160 and a portion of the peripheral portion 408 on the second side 404 of the power changing lens 153 can come to rest on a plurality of support surfaces 170. The support surfaces 170 can extend radially inward from the posterior end of compression arms 180. Each support surface 170 can have an outer end 172 coupled with a posterior end of a corresponding compression arm 180 and an inner end 174 disposed radially inwardly of the outer end 172 (see FIGS. 6 and 6A).

The circumferential extent of the support surfaces 170 can be the same at each of a plurality of spaced apart locations. The circumferential extent can extend over an arc of approximately 25 degrees, over an arc of approximately 20, over approximately an arc of 15 degrees, over an arc of approximately 10 degrees, or over an arc in a range of approximately 10-30 degrees, or over an arc in a range of approximately 15-20 degrees. The radial extent of the support surfaces 170 can be approximately 2-20% of the diameter of the second side 404 of the power changing lens 153. In other embodiments the radial extent of the support surfaces 170 can be approximately 4-15%, 6-10%, or about 8% of the diameter of the second side 404 of the power changing lens 153.

Preferably at least three of the support surfaces 170 are coplanar with each other. Preferably at least three of the support surfaces 170 are aligned in a common plane that is substantially transverse to, e.g., within about 2-5 degrees of perpendicular to, the optical axis OA of the accommodating IOL device 150. In one embodiment three or more, e.g., all, of the support surfaces 170 are aligned in a plane perpendicular to the optical axis OA. In some cases, the support surfaces 170 are configured to contact the second side 404 of the power changing lens 153 and when in such contact to cause the optical axis of the power changing lens 153 to be less than 25 degrees offset form the optical axis of the base lens 163. The support surfaces 170 can be configured to contact the second side 404 of the power changing lens 153 and when in such contact to cause the optical axis of the power changing lens 153 to be less than 15, less than 10, less than 5 or less than 3 degrees offset form the optical axis of the base lens 163. In various embodiments, the edges of the haptic 165 and/or the base lens 163 can be rounded to reduce or mitigate the occurrence of dysphotopsia. For example, one or more edges in the optical path can be configured as rounded edges instead of sharp edges to reduce or mitigate dysphotopsia. As another example, the edges of the lens retention portion 164, the edges of the equator contact segments 141, the edges of one or more support surfaces 170 can be at least partially configured as rounded edges instead of sharp edges to reduce or mitigate dysphotopsia. Without any loss of generality, a plurality of the edges in a circular region of diameter 7 mm around a geometric center of the IOL device 150 can be configured as rounded edges instead of sharp edges to reduce or mitigate dysphotopsia.

Although the base member 151 is illustrated to have six support surfaces 170, there could be fewer or more than six support surfaces 170. In various embodiments there are four, three or two support surfaces 170 against which the power changing lens 153 is placed to position the power changing lens 153 in the base member 151.

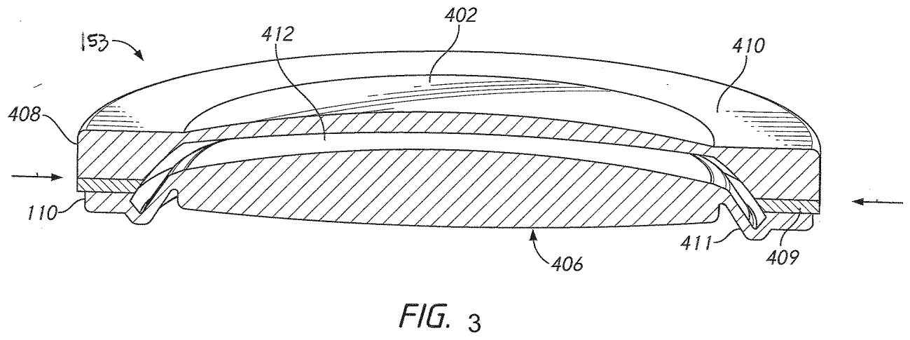

FIGS. 2A-2D and 3 illustrate the power changing lens 153 in detail. The power changing lens 153 includes a flexible membrane 402, an optic 406 and an outer circumference 409, which may be referred to as a circumferential peripheral edge. The outer circumference 409 couples the flexible membrane 402 to the optic 406. A membrane coupler 410 is disposed from the outer circumference 409 to couple the flexible membrane 402 with the outer circumference 409. Similarly, an optic coupler 411 is disposed from outer circumference 409 to couple the optic coupler 411 with the outer circumference 409. Preferably, the optic coupler 411 is angled toward the flexible membrane 402 such that it positions the optic 406 toward the flexible membrane 102.

The structure of the power changing lens 153 is simplified by not requiring any traditional elongate thin haptic structures. Rather the peripheral portion 108 is formed as an annulus. The axisymmetric structure enables the power changing lens 153 to be positioned in any rotational position within the cavity 160 in embodiments without cylinder power on the optic 106. Any rotational position of the power changing lens 153 in the base member 151 will provide uniform compression and such compression will provide uniform power change primarily by changing the shape of the flexible membrane 402. The power changing lens 153 provides a fluid filled lens with one membrane. The optic 406 is a moving Optic. The power changing lens 153 changes power through diametrical compression of the peripheral portion 408 in response to ocular forces. Such forces deflect the flexible membrane 102 as indicated by the dashed line anterior of (above) the solid line position of the flexible membrane 402 in FIG. 3. The optic 406 also moves in response to compression of the peripheral portion 408 as indicated by the dash line anterior of (above) the optic 406 in FIG. 3, Without subscribing to any particular theory, the uniformity of the power change can be measured using a bench-top measurement system. The bench-top measurement system can comprise a cylindrical device that can hold the IOL device 150 including the base member 151 and the power changing lens 153 in a compressed state similar to the accommodated state in the eye of the patient. The amount of compressive force applied by the cylindrical device can be sufficient to achieve a power change equivalent to an optical power of 4.0 Diopter in the IOL plane. The power change of the IOL device 150 can be considered to be uniform if the average optical power measured along any of the transverse axes is between 3.0 Diopter and 5.0 Diopter in the IOL plane.

The optic 406 is not a major or main driving force in the change in shape of the flexible membrane 402. Rather, the optic 406 follows the movement of the flexible membrane 402 in response to shifting of the fluid in the closed cavity 412. The optic 406 can be considered to be floating on the fluid in the closed cavity 412 and thus anterior movement of the fluid in response to ocular forces causing compression of the peripheral portion 408 as indicated by arrows A allows the optic 406 to shift anteriorly. Posterior movement of the fluid in response to relaxation of the peripheral portion 408 as indicated by removal of ocular forces in a direction opposite arrows A allows the optic 406 to shift posteriorly. The shifting of the fluid and the optic 406 minimizes distortion of the power changing lens 153 and thus minimizes any dysphotopsia and any other optical interference during power change. The arrows shown within the cross-section in FIG. 3 are intended to show the compression force F divided into components in the power changing lens 153. The vast majority of the force F is driven into the flexible membrane 402 due to the membrane being in the plane of the equator contact segments 141 in the posterior segment 163 of the haptic 165. This is due to the deep set position of the power changing lens 153 in the base member 151. Some force may be transferred into the optic coupler 411. However, a response to this force can be articulating the coupler rather than directly moving the optic 406 forwardly. Thus even the force distribution within the power changing lens 153 attenuates anterior movement driven in response to the compression force F.

The configuration of the power changing lens 153 to enable the optic 106 to follow anterior shape change of the flexible membrane 402 enables the posterior surface of the optic 406 to be placed adjacent to the anterior surface 164 of the base lens 163. The distance between these structures can be 0.5 mm or less, can be 0.4 mm or less, can be 0.3 mm or less, can be about 0.2 mm, or can be 0.2 mm or less. The close positioning of these structures enables the deep inset position of the power changing lens 153 in the base member 151.

In some embodiment the performance of the power changing lens 153 is dependent on placing the power changing lens 153 in the eye such that the flexible membrane 402 is anterior of the optic 406. Also, the manner in which the power changing lens 153 is compressed for insertion into the eye can be critical to successful delivery into the eye. Certain variants aid quickly confirming the orientation of the power changing lens 153.

FIG. 3 shows an optional additional visible color structure 409 that can provide confirmation of the orientation of the power changing lens 153, e.g., to positively identify the location of the flexible membrane 402 and the optic 406. The power changing lens 153 can have a visible color structure 409 disposed in the peripheral portion 408. The visible color structure 409 has an at least partially opaque dye or pigment. The opaque dye or pigment can be any color, which can include red, orange, yellow, green, blue, indigo, violet, and/or any other suitable color or combination of colors. The visible color structure 409 can be a variety of cross-sectional sizes and shapes, which can be continuous or varied. For example, the visible color structure 409 can be a complete annulus that is visible from a peripheral, an anterior and/or a posterior side. The visible color structure 409 can include one or a plurality of arcs or arc segments visible from a peripheral, an anterior and/or a posterior side. The visible color structure 409 is disposed between an anterior portion and posterior portion of the peripheral portion 408 such that the at least partially opaque dye or pigment of the visible color structure 409 is contained in the power changing lens 153 and positioned radially outward of an optical axis A and in some cases outward of a closed cavity 412 of the lens 153. The visible color structure 409 is disposed between a first side 400 (anterior side) and a second side 404 (posterior side) of the power changing lens 153. The visible color structure 409 is disposed closer to the posterior portion than to the anterior portion of the peripheral portion 408 in one example. This positioning enables convenient visual verification of the orientation of the power changing lens 153. The visible color structure 409 is positioned closer to a plane tangential to the posterior surface of the optic 406 than to a plane tangential to an anterior surface of the flexible membrane 402. Accordingly, when viewed from the side, the side of the power changing lens 153 that is closest to the visible color structure 409 is the side of the optic 406, e,g., the second side 404, while the side that is farthest from the visible color structure 409 is the side of the flexible membrane 402, e.g., the first side 400.

The visible color structure 409 can provide a visual verification that the power changing lens 153 is loaded correctly into a injector. In some aspects, the visible color structure 409 can be used to visually verify that the power changing lens 153 is secured within the base member 151 by the lens retention portions 164. For example, the visible color structure 409 can be a continuous annular shape that is visually disrupted, when viewed from above, by the lens retention portions 164 (if the lens retention portions 164 are opaque or have a solid color, as described elsewhere herein) when the power changing lens 153 is secured within the base member 151 by the lens retention portions 164. Accordingly, the power changing lens 153 is secured by a given lens retention portion 164 when the visible color structure 409 is disrupted, when viewed from above, at the position of the given lens retention portion 164. Relatedly, the power changing lens 153 is not secured by a given lens retention portion 164 when the visible color structure 409 is not disrupted, when viewed from above, at the position of the given lens retention portion 164.

In some aspects, the visible color structure 409 can be combined with an adhesive that joins the anterior portion and posterior portion of the peripheral portion 408. The adhesive can be the same material as the power changing lens 153 or another suitable material. In some aspects, the visible color structure 409 is rotationally symmetrically disposed about the optical axis. The visible color structure 409 can be an arcuate band surrounding the optical axis. In some aspects, the visible color structure 409 reduces observable glare transmitted through the peripheral portion 408.

### Lens Oil

In accordance with several implementations, the lens oil may comprise a silicone oil terpolymer represented by Formula (I):

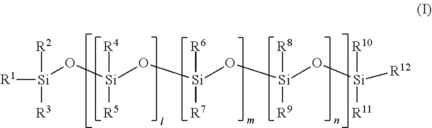

wherein R¹ and R¹² are independently selected from optionally substituted alkyl or optionally substituted alkenyl including vinyl(alkyl); each of R², R³, R¹⁰ and R¹¹ may be independently hydrogen or optionally substituted alkyl; R⁴ is optionally substituted haloalkyl; R⁵ is optionally substituted alkyl or optionally substituted haloalkyl; R⁶ is optionally substituted aryl or optionally substituted aryl(alkyl); R⁷ is optionally substituted aryl, optionally substituted aryl(alkyl), or optionally substituted alkyl; each of R⁸ and R⁹ are independently optionally substituted alkyl; 1 is a molar fraction of 0.01 to 0.8; m is a molar fraction of 0.01 to 0.5; and n is a molar fraction of 0.01 to 0.6. The molar fractions or percentages are for just the three types of blocks; the end blockers, catalyst and any other components in the reaction and/or end product polymer are excluded from the calculation of molar fractions or percentages. As noted above, the formula above refers to a random block copolymer in which the blocks are made up of multiple —Si(R^{x},R^{x})O— silicone units of a particular type combined randomly with other blocks of different types. The structure above is a formula rather than a literal structure of the polymer; the polymer does not consist of only three large blocks in the order shown.

In some implementations, the alkyl and/or alkylene groups have 1 to 20 carbons, including 1 to 6 carbons and 1 to 4 carbons. Preferred alkyl groups include, but are not limited to, methyl, ethyl, and propyl. Preferred alkenyl groups include vinyl, vinylmethyl, and vinylethyl. Preferred aryl and aryl(alkyl) groups include phenyl, and phenylmethyl. In some implementations, the mole fractions of the three components of the terpolymer are as follows: the mole fraction for 1 is about 0.01 to 0.8, including about 0.1 to 0.7, about 0.4 to 0.6, about 0.45, about 0.5, and about 0.55; the mole fraction for m is about 0.01 to 0.5, including about 0.05 to 0.3, about 0.5 to 0.2, about 0.1, and about 0.15; and the mole fraction for n is about 0.01 to 0.6, including about 0.1 to 0.5, about 0.2 to 0.5, about 0.3, about 0.35, about 0.4 and about 0.45. In some implementations, for R¹ to R³ and R⁶ to R¹², if one or more groups are substituted, the substitution is with a group other than halo or halogen. In certain implementations, only one or two optionally substituted groups are actually substituted and in other implementations no optionally substituted groups are actually substituted. In certain preferred implementations, haloalkyl is fluoroalkyl, including trifluoroalkyl such as trifluoroethyl, or trifluoropropyl. Some implementations include two or more of the foregoing.

In some implementations, R¹ and R¹² are methyl, ethyl, or vinyl.

In some implementations, R², R³, R¹⁰ and R¹¹ are C₁₋₆ alkyl, including methyl or ethyl.

In some implementations, R⁴ is C₁₋₆ fluoroalkyl, including di- and tri-fluoroalkyl groups. Certain implementations comprise R⁴ as 2,2,2-trifluoroethyl or 3,3,3-trifluoropropyl.

In some implementations, R⁵ is C₁₋₆ haloalkyl, including fluoroalkyl. It may be the same as R⁴, or it may be different. In other implementations, R⁵ is C₁₋₆ alkyl, including methyl or ethyl.

In some implementations, R⁶ is phenyl.

In some implementations, R⁷ is phenyl or C₁₋₆ alkyl, including methyl or ethyl. In some implementations, R⁶ and R⁷ are the same group.

In some implementations, R⁸ and R⁹ are independently C₁₋₆ alkyl, including methyl or ethyl. In some implementations, R⁸ and R⁹ are the same group.

In some implementations, 1 is 0.4 to 0.6, including 0.5; m is 0.05 to 0.15, including 0.1; and/or n is 0.3 to 0.5, including 0.4.

The silicone lens oil of Formula (I) may further have one or more or the physical and/or chemical properties described below. A lens oil need not have any of these properties to be within the scope of this disclosure.

Molecular Weight The silicone oil may have an average molecular weight (M_{w}) of about 1000 to 30,000 Daltons, including about 1000 to 25,000 Daltons, about 10,000 to 30,000 Daltons. about 15,000 to 30,000 Daltons, about 10,000 to 25,000 Daltons, about 1000 to 15,000 Daltons, about 1000 to 15,000 Daltons. about 1000 to 10,000 Daltons, about 2500 to 10,000 Daltons, about 2500 to 7500 Daltons, about 3000 to 7500 Daltons, about 4000 to 7000 Daltons. about 4500 to 6500 Daltons, and about 5000 to 6500 Daltons. The silicone oil may have an average molecular weight (M_{w}) of about 5500+/−300 Daltons, about 5600+/−300 Daltons, about 5700+/−300 Daltons, about 5800+/−300 Daltons, about 5900+/−300 Daltons, about 6000+/−300 Daltons. about 6100+/−300 Daltons, about 6200+/−300 Daltons, about 6300+/−300 Daltons, about 6400+/−300 Daltons, about 6500+/−300 Daltons, about 6600+/−300 Daltons, about 6700+/−300 Daltons, about 6800+/−300 Daltons, about 6900+/−300 Daltons, or about 7000+/−300 Daltons. in another implementation, the silicone oil may have an average molecular weight (MN) of about 25,000+/−1000 Daltons, about 26,000+/−1000 Daltons, about 27,000+/−1000 Daltons, about 28,000+/−1000 Daltons, about 29,000+/−1000 Daltons, or about 30,000+/−1000 Daltons. In another implementation, the silicone oil may have an average molecular weight (M_{w}) of about 10,000+/−1000 Daltons, about 12,000+/−1000 Daltons, about 15,000+/−1000 Daltons, about 17,000+/−1000 Daltons, about 20,000+/−1000 Daltons, or about 22,000 +/−1000 Daltons. In another aspect, the silicone oil may have a mean molecular weight within a range that includes any two of the foregoing values.

In some implementations, including but not limited to those having the weight average molecular weights noted above, the lower end of the molecular weight distribution may be no lower than about 500 Daltons, about 1000 Daltons, about 2000 Daltons, about 2500 Daltons, about 3000 Daltons, about 4000 Daltons, about 5000 Daltons, about 6000 Daltons, about 7000 Daltons, about 8000 Daltons, about 9000 Daltons, or about 10,000 Daltons. Low molecular weight polymers have a greater ability to permeate a solid silicone material. Such solid silicone materials include components of the IOLs as described in the Appendix and otherwise. It has been discovered that silicone oils having a greater methyl content (i.e. more of R⁴ to R⁹ are methyl) have a greater permeability, such that silicone oils having a lower methyl content or no methyl content may include molecules having a lower molecular weight and thus a lower end of the distribution than those having higher methyl content.

Viscosity The silicone lens oil may have a viscosity in a range from about 1 to 3000 cP at 25° C., including about 5 to 1000 cP at 25° C., about 5 to 500 cP at 25° C., and about 20 to 250 cP at 25° C. The lens oil may have a viscosity outside these ranges as well.

Index of Refraction In some implementations, the silicone lens oil has have an index of refraction in a range from about 1.40 to about 1.60, including about 1.41, 1.42, 1.43, 1.44, 1.45, 1.46, 1.47, 1.48, 1.49, 1.50, 1.51, 1.52, 1.53, 1.54, 1.55, 1.56, 1.57, 1.58. 1.59. including values in between the listed values such as 1.465, 1.4452, 1.455, 1.445, and the like.

The silicone lens oil disclosed herein in some aspects may be substantially index-matched to the bulk material(s) that form the IOL device in which it is used. As used herein, an index-matched material refers to a material whose index of refraction that is about equal to, or closely approximates, the index of refraction of another material.

Surface Tension In preferred implementations, the silicone lens oil has a surface tension sufficiently high or sufficiently different enough from the inner surface of the cavity such that when it is placed into the cavity of the IOL it is repelled by the surface of the material that makes up the shell or walls of the cavity (if the walls of the cavity have little or no fluorine content), which may be made of a silicone including a non-halogenated silicone. This helps the lens cavity to take on a biconvex shape when fluid is introduced. If the surface tension of the silicone lens oil is not sufficiently high (or different enough from the cavity walls), a dimple or concave shape may form on one or both sides of the lens providing for an optic of lesser or poor quality.

The surface tension may be manipulated by both the type and quantity of monomers used to form the polymer, which determines the type and mole fraction of silicone units that form the various blocks in the polymer itself. It has been found that inclusion of silicone units that include a fluorine-containing group tend to increase the surface tension. Phenyl groups are also of benefit to increase surface tension. The table below illustrates the surface tension measured for various types of materials. a difference in surface tension between the fluid and the cavity wall of at least 0.5 to 1.0 mN/m or higher is preferred, including from 0.5 to 7 mN/m and from 1.0 to 5 mN/m.

TABLE 1

| | | Surface Tension | Surface Tension |
|---|---|---|---|
| Sample | Silicone Unit Content | 25° C. | 40° C. |
| ID | (mole percent) | (mN/m) | (mN/m) |
| 1 | 25% DP, 75% DM | 21.01 ± 0.07 | 20.18 ± 0.04 |
| 2 | 10% DP, 40% DM, 50% TF | 22.88 ± 0.01 | 21.97 ± 0.03 |
| 3 | 100% TF | 23.65 ± 0.04 | 23.00 ± 0.08 |
| 4 | 15% DP, 85% TF | 24.02 ± 0.08 | 23.19 ± 0.16 |

In Table 1 above, DM is a dimethyl silicone unit (—Si(CH₃)₂O—), DP is a diphenyl silicone unit (—Si(C₆H₅)₂O—) and TF is a methyl, 3,3,3-trifluoropropyl methyl silicone unit (—Si(CH₃)(CH₂CH₂CF₃)O—). The surface tension of the samples was determined in duplicate at 24.9° C. and 39.9° C. using a Wilhelmy plate on a DCAT 25 tensiometer equipped with a temperature control system.

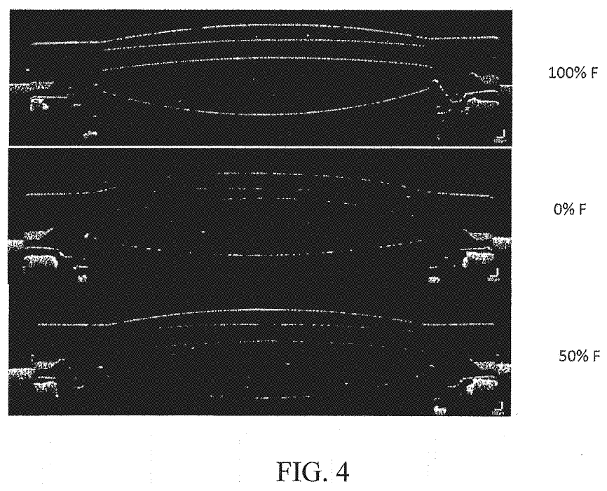

The difference in surface tension or surface energy is further illustrated by the optical coherence tomography (OCT) images in FIG. 4 The images are cross-section views of three IOLs made from diphenyl and dimethyl siloxanes that are identical except for the lens oil that is placed in the space between the second and third lines when starting at the top of each image. The lens oil in the top image has a silicone backbone that is solely 3,3,3-trifluoropropyl methyl siloxane (100% F), the center image has a backbone that is solely dimethyl and diphenyl siloxanes (0% F), and the bottom image is 50 mole % 3,3,3-trifluoropropyl methyl siloxane, 40 mole % dimethyl siloxane and 10 mole % diphenyl siloxane (50% F). The OCT images show that the second line is deflected away from the third line in the 100% and 50% fluorinated, whereas in the second image having no fluorinated substituents, the second line bends toward the third line, resulting in a less convex or even slightly dimpled or concave lens surface. Dimpled, flattened or concave surfaces, as in the center image, are less suitable for a lens as compared to the top and bottom images having a more convex surface, that is one that is curved up towards the first (top) line. Lens oils according to some implementations that are useful in making IOLs may have one or more of the following characteristics: highly hydrophobic, very low surface energy, and a repulsive surface tension.

Crosslinking Although some implementations of the silicone lens oil may include cross-linkable terminal groups such as vinyl, the lens oil is preferably not crosslinked. The uncrosslinked lens oil is a fluid that is incompressible or substantially incompressible and thus as fluids do not have a Poisson ratio or a Young's modulus, unlike silicone polymers that are even lightly crosslinked or gelled which behave mechanically with a Poisson effect. Fluid mechanical behaviour is instead affected by viscosity and shear and depend on the type of rheology exhibited by that fluid such as Newtonian, or non-Newtonian fluid behaviour. In preferred implementations, the polymer chains that make up the silicone oil do not diffuse through the bulk polymer material of the IOL device despite not being crosslinked or gelled.

### Methods of Preparation

The silicone lens oil disclosed herein may be synthesized using known synthesis and polymer chemistry techniques. For instance, methods of making the silicone oil may include anionic addition polymerization, living polymerization, living anionic polymerization, and the like. In some implementations, the oils are made by methods described below, or by modification of these methods. Starting materials can be obtained commercially and/or prepared utilizing known synthetic procedures. A general synthetic route to one example of a silicone oil of Formula (I) is shown and described herein. The starting materials and route shown and described herein are illustrative only and are not intended, nor are they to be construed, to limit the scope of the disclosure and/or claims in any manner whatsoever. Those skilled in the art will be able to recognize modifications of the disclosed syntheses and to devise alternate routes based on the disclosures herein; all such modifications and alternate routes are within the scope of the claims.

The presently disclosed silicone oil may be manufactured by a simple polymerization process. The starting materials include a cyclic siloxane corresponding to each block type of the terpolymer, a chain growth terminator, and a catalyst. The starting materials other than the catalyst are weighed out and placed into the reaction flask, which may be a suitably-sized round-bottom flask equipped with a condenser having cool water or other coolant circulating therethrough. The contents of the flask are stirred with heating to about 100+/−10° C., with the catalyst being added when the temperature reaches the lower end of that temperature range. The temperature of the reaction mixture is maintained in the about 100+/−10° C. temperature range for approximately 2 hours during which the temperature should not be allowed to exceed a temperature in which the catalyst will deactivate or one of the starting materials will degrade or decompose. The polymerization reaction has occurred once the contents of the flask turn from a milky-white to clear. An increase in the viscosity of the contents may also become apparent. The mixture should continue to be stirred at 100+/−10° C. for at least 2 hours (including at least 3 hours, at least 4 hours and from 2 to 4 hours) to allow the mixture to reach equilibrium. The temperature of the mixture is then increased to about 150° C. to deactivate the catalyst. Once the temperature reaches about 150° C., the heating is shut off or removed but stirring is continued, and the mixture is allowed to cool down until it reaches about 100° C. or less. The stirring may be discontinued once the temperature of the contents, now a raw polymer mixture, is below about 100° C.

The raw polymer mixture may then be purified by any suitable method for polymers, including but not limited to supercritical fluid extraction, use of vacuum distillation such as by a thin film/wiped film evaporator, and size exclusion chromatography/gel permeation chromatography (SEC)/(GPC). These techniques can be used to remove residual monomers or other starting materials and low molecular weight components that may be undesirable in the finished product. In some implementations, two or more methods may be used to purify the silicone oil. Purification of the silicone oil helps to reduce permeation of components into and/or through the walls of the lens device and/or reduces haze in the final lens product. Purification also provides for a more stable index of refraction in the purified lens oil and may increase the surface tension as compared to unpurified material.

Vacuum distillation may be performed using a commercially available thin film or wiped film evaporator. SEC/GPC is a technique that utilizes a column packed with porous crosslinked gels to separate polymer molecules according to their retention time in the column based upon molecular size.

In the case of supercritical fluid extraction, this may be performed using any of a variety of commercially available extraction units using supercritical CO₂, propane, ethane, ethylene, combinations thereof, and/or other suitable eluting solvent as would be appreciated by skilled artisans upon reading the present disclosure. In certain implementations, a solvent such as an alcohol (e.g. ethanol, isopropyl alcohol) may be added to the silicone oil prior to extraction at an amount of about 10-40% by weight, including about 25-30% by weight. Not intending to be bound by theory, it is thought that the addition of the alcohol may help to remove small amounts of polar materials that, if left in the oil, may complex with water vapor which could contribute to cloudiness in a finished lens product. For example, when a mixture of 70% silicone oil/30% IPA (w/w) is extracted with supercritical CO₂, the IPA is among the first fractions removed from the raffinate, which is the product silicone oil.

The starting materials may be cyclic tri- or tetra-siloxanes including, but not limited to, octaphenylcyclotetrasiloxane, hexamethylcyclotrisiloxane, octamethylcyclotetrasiloxane, (2,2,2-trifluoroethyl)methylcyclotrisiloxane, and (3,3,3-trifluoropropyl)methylcyclotrisiloxane. Chain growth terminators include trimethylsiloxy-terminated polydimethylsiloxane (e.g. 2 cSt viscosity CAS No. 9016-00-6/63148-62-9, product code DMS-T02 from Gelest) or vinyl-terminated polydimethylsiloxane (e.g, 2-3 cSt viscosity, CAS No. 68083-19-2, product code DMS-V03 from Gelest). Suitable catalysts include tetramethylammonium siloxanolate.

### EXAMPLES

Twelve examples of silicone lens oil (E1 to E12) according to Formula (I) of the present disclosure were synthesized and analyzed according to one implementation of a synthetic method. The procedure for E1 is presented below as an example of the procedure.

The starting materials, (3,3,3-trifluoropropyl)methylcyclotrisiloxane, octaphenylcyclotetrasiloxane, and octamethylcyclotetrasiloxane, were added to a one liter round bottom flask in amounts corresponding to the mole percentages provided in the tables below. As noted above, the mole percentages of the siloxanes which form the repeating blocks of the polymers add to 100%; the endblocker, catalyst and other components are not included in the total of 100%.

TABLE 2

| COMPONENT | QUANTITY | MOLE % |
|---|---|---|
| (3,3,3-trifluoropropyl)methylcyclotrisiloxane | 418 g | 50 |
| Octaphenylcyclotetrasiloxane | 106.1 g | 10 |
| Octamethylcyclotetrasiloxane | 158.6 g | 40 |
| Trimethyl endblocked polydimethyl siloxane | 17.4 g | (0.57 not |
| | | included |
| | | in mole % |
| | | total) |
| Tetramethylammonium Siloxanolate | 11 g | (catalyst) |

The trimethylsiloxy-terminated polydimethylsiloxane chain growth terminator (product code DMS-102 from Gelest) was added to the flask which was fitted with a condenser with circulating cool water and a mechanical stirrer and placed into a heating mantle. The heating of the mixture was initiated, and 0.25 pph of tetramethylammonium siloxanolate catalyst was added when the temperature reached 90° C. The temperature of the mixture was kept at 100+/−10° C. (e.g. 97.8° C.) until the mixture became clear with an increase in viscosity. Care was taken to keep the temperature under 115° C. to avoid deactivation of the catalyst during this initial stage.

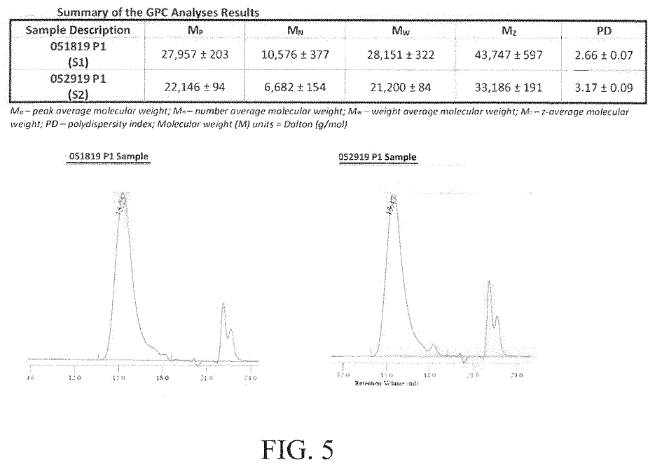

Mixing was continued for at least two hours at 100+/−10° C., after which the temperature was increased to about 150° C. Once the temperature reached about 150° C., the heating was shut off and mixing continued until the temperature had decreased to about 100° C. or less. The product was then cleaned up first by distillation with a wipe film evaporator, followed by supercritical fluid extraction with CO₂. The polymer was then transferred to a suitable storage container and tested for molecular weight and refractive index. FIG. 5 includes the results of analysis of the molecular weight distribution of the two samples of polymer by GPC. This method was used to determine the molecular weights in this disclosure.

The results of the refractive index testing and the appearance of E1 and each of the other 11 experiments is presented in the table below:

TABLE 3

| | Tri F, methyl | Diphenyl | Dimethyl | Refractive | |
|---|---|---|---|---|---|
| | (mole %) | (mole %) | (mole %) | Index | Appearance |
| E1 | 50 | 10 | 40 | 1.42 | clear |
| E2 | 50 | 30 | 20 | 1.47 | clear |
| E3 | 50 | 30 | 20 | 1.47 | clear |
| E4 | 50 | 15 | 35 | 1.435 | clear |
| E5 | 50 | 25 | 25 | 1.465 | clear |
| E6 | 75 | 25 | 0 | 1.455 | clear |
| E7 | 70 | 30 | 0 | 1.478 | clear |
| E8 | 50 | 15 | 35 | 1.441 | clear |
| E9 | 50 | 25 | 25 | 1.465 | clear |
| E10 | 70 | 30 | 0 | 1.475 | clear |
| E11 | 60 | 40 | 0 | 1.498 | clear |
| E12 | 50 | 15 | 35 | 1.44 | clear |

The invention described and claimed herein is not to be limited in scope by the specific implementations, aspects, and embodiments disclosed herein. Indeed, various modifications of the invention in addition to those shown and described herein will become apparent to those skilled in the art from the foregoing description. Such modifications are also intended to fall within the scope of the appended claims.

It is to be understood that the term “embodiment” as used herein refers to an aspect or implementation of the invention disclosed herein, and that embodiments may be combined with one another.

While certain implementations and embodiments of the inventions have been described, these have been presented by way of example only, and are not intended to limit the scope of the disclosure, indeed, the novel compositions and devices described herein may be embodied in a variety of other forms. Furthermore, various omissions, substitutions and changes in the materials and devices described herein may be made without departing from the spirit of the disclosure. The accompanying claims and their equivalents are intended to cover such forms or modifications as would fall within the scope and spirit of the disclosure. Accordingly, the scope of the inventions are defined only by reference to the appended claims and other claims that may be sought in the future.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying appendix, claims, abstract and drawings), and/or all of the steps of any method so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations or embodiments can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may he embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, I degree, or 0.1 degree.

Unless the context requires otherwise, use of the word “comprise” and variations thereof, such as, “comprises” and “comprising” in the description and claims is open ended and synonymous with “including” or “including but not limited to” and intended to also include the narrower terms “consisting of” and “consisting essentially of,” the latter term meaning that the scope is limited to the recited elements or steps and any others that do not materially affect the basic and novel characteristics of what is already recited.

Unless otherwise defined, all terms (including technical and scientific terms) are to be given their ordinary and customary meaning to a person of ordinary skill in the art and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not he utilized in a particular embodiment of the invention. Likewise, a group of items linked with the conjunction “and” should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a recitation of a numeric range is provided, it is intended to serve as a shorthand notation of referring individually to each separate value falling within the range inclusive of the values defining the range, and each separate value is incorporated in the specification as it were individually recited herein. Further, it is understood that the upper and lower limits are encompassed within the recited range.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” and the definite article “the” do not exclude a plurality unless context clearly dictates otherwise. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc,” is used, in general such a construction is intended in the sense one having skill in the art would understand the convention, e.g., as including any combination of the listed items, including single members (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, Whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

All references cited herein are incorporated herein by reference in their entirety. To the extent publications and patents or patent applications incorporated by reference contradict the disclosure contained in the specification, the specification is intended to supersede and/or take precedence over any such contradictory material.

Headings are included herein for reference and to aid in locating various sections. These headings are not intended to limit the scope of the concepts described with respect thereto. Such concepts may have applicability throughout the entire specification.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A terpolymer represented by the following Formula (I): 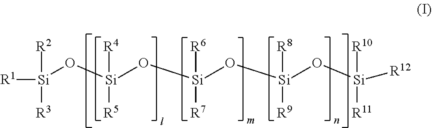
wherein the polymer is not crosslinked, and
R¹ and R¹² are independently selected from optionally substituted alkyl or optionally substituted alkenyl;
R², R³, R¹⁰ and R¹¹ are independently selected from hydrogen or optionally substituted alkyl;
R⁴ is optionally substituted haloalkyl;
R⁵ is optionally substituted alkyl or optionally substituted haloalkyl;
R⁶ is optionally substituted aryl or optionally substituted aryl(alkyl);
R⁷ is optionally substituted aryl, optionally substituted aryl(alkyl), or optionally substituted alkyl;
R⁸ and R⁹ are independently optionally substituted alkyl;
1 is a molar fraction of 0.01 to 0.8;
m is a molar fraction of 0.01 to 0.5; and
n is a molar fraction of 0.01 to 0.6.

2. The terpolymer of claim 1, wherein R¹ and R¹² are methyl or vinyl.

3. The terpolymer of claim 1, wherein R², R³, R¹⁰ and R¹¹ are methyl or ethyl.

4. The terpolymer of claim 1, wherein R⁴ is fluoroalkyl.

5. The terpolymer of claim 1, wherein R⁴ is 2,2,2-trifluoroethyl or 3,3,3-trifluoropropyl.

6. The terpolymer of claim 1, wherein R⁵ is methyl or ethyl.

7. The terpolymer of claim 1, wherein R⁶ is phenyl.

8. The terpolymer of claim 1, wherein R⁷ is phenyl, methyl or ethyl.

9. The terpolymer of claim 1, wherein R⁸ and R⁹ are independently methyl or ethyl.

10. The terpolymer of claim 1, wherein 1 is 0.4 to 0.6.

11. The terpolymer of claim 1, wherein m is 0.05 to 0.15.

12. The terpolymer of claim 1, wherein n is 0.3 to 0.5.

13. (canceled)

14. (canceled)

15. (canceled)

16. (canceled)

17. (canceled)

18. (canceled)

19. (canceled)

20. An accommodating IOL, comprising:
a base lens;
a haptic; and
a power changing lens, the power changing lens comprising a first side, a second side, a peripheral portion coupling the first and second sides, and a closed cavity configured to house a fluid according to claim 1.
wherein the first side of the power changing lens is spaced from a first edge of the haptic.

21. The accommodating IOL of claim 20, wherein the haptic comprises:
a first open end;
a second end coupled with the base lens; and
an outer periphery configured to engage an equatorial region of a capsular bag;
an inner periphery and a height between a first edge and a second edge, the inner periphery disposed about a cavity and having a lens retention portion configured to receive and retain a lens.

22. The accommodating IOL of claim 21, wherein the power changing lens is configured to fit within the cavity with the first side of the power changing lens spaced apart from the first edge of the haptic.

23. The accommodating IOL of claim 20, wherein at least a portion of the haptic comprises a material with high contrast to the material of the peripheral portion of the power changing lens.

24. The accommodating IOL of claim 20, wherein at least a portion of the haptic comprises a first color surface and the peripheral portion of the power changing lens comprises a second color surface visually distinct from the first color surface.

25. The accommodating IOL of claim 20, wherein a plurality of open channels extend from outside of the accommodating IOL to a space between the base lens and the second side of the power changing lens.

26. The accommodating IOL of claim 20, wherein the haptic comprises a plurality of spaced apart radial hinges comprising a first portion coupled with the base lens and a second portion coupled with the haptic, a gap being provided between the radial hinges and the second side of the power changing lens.

27. The terpolymer of claim 1, wherein:
R¹¹ and R¹² are methyl or vinyl;
R², R³, R¹⁰ and R¹¹ are methyl or ethyl;
R⁴ is 2,2,2-trifluoroethyl or 3,3,3-trifluoropropyl;
R⁵ is methyl or ethyl;
R⁶ is phenyl;
R⁷ is phenyl, methyl or ethyl;
R⁸ and R⁹ are independently methyl or ethyl;
1 is 0.4 to 0.6;
m is 0.05 to 0.15; and
n is 0.3 to 0.5.
